(19) United States
(12) Patent Application Publication
(10) Pub. No.: US 2022/0016446 A1
(43) Pub. Date: Jan. 20, 2022

(54) Delivering an Airflow to a User

(71) Applicant: X Development LLC, Mountain View, CA (US)

(72) Inventors: Faust Whale, San Francisco, CA (US); Radu Gogoana, Burlingame, CA (US); Philip Edwin Watson, Santa Cruz, CA (US)

(21) Appl. No.: 16/928,937

(22) Filed: Jul. 14, 2020

(51) Int. Cl. A62B 7/10

(52) U.S. Cl. CPC A62B 7/10

(57) ABSTRACT

A method of delivering air to a user includes receiving, by a controller and from a sensor, motion information of the user. The motion information represents motion of a portion of a head of the user below eyes of the user. The sensor and the controller are coupled to a portable article of the user. The method also includes determining, by the controller, based on the motion information, a position of the head of the user. The method further includes controlling, by the controller based on the determined position, a fluid outlet of a fluid conduit fluidly coupled to and configured to receive air from a fan.

### TECHNICAL FIELD

This disclosure relates to air delivery systems, and more particularly to individual air delivery systems.

### BACKGROUND

Air can contain airborne particles that are harmful to the human body. For example, air can contain pollutants, virus particles, and respiratory allergens that affect people in multiple ways. Individual air delivery systems can deliver clean air to a user to protect the user from such airborne particles. Improvements to individual air delivery systems are sought.

### SUMMARY

In an example implementation, a method of delivering air to a user includes receiving, by a controller and from a sensor, motion information of the user. The motion information represents motion of a portion of a head of the user below eyes of the user. The sensor and the controller are coupled to a portable article of the user. The method also includes determining, by the controller, based on the motion information, a position of the head of the user. The method further includes controlling, by the controller based on the determined position, a fluid outlet of a fluid conduit fluidly coupled to and configured to receive air from a fan. The fan is coupled to the portable article of the user.

In some implementations, receiving the motion information includes receiving the motion information from a motion detector configured to sense an orientation of the jaw of the user.

In some implementations, controlling the fluid outlet includes controlling vents of the fluid outlet to change a direction of air flowing from the fluid outlet.

In some implementations, the method further includes receiving, from one or more sensors, ambient condition information representing at least one of 1) wind speed information, 2) wind direction information, or 3) airborne particulate information. The method also includes controlling, by the controller, based on the ambient condition information, at least one of the fluid outlet and the fan. In some implementations, the method also includes receiving, from the one or more sensors, breathing information of the user, and determining, by the controller and based on the breathing information, a time of inhalation of the user. The method also includes controlling, by the controller, based on the ambient condition information and on the time of inhalation, at least one of the fluid outlet and the fan.

In some implementations, the method also includes receiving, from one or more sensors, facial information of the user representing facial features of the user below the eyes of the user. The method also includes determining, by the controller, based on the facial information of the user and on a mathematical model of the Coand\check{a} effect, a location of a facial region of the user away from the nose and mouth of the user, and controlling, by the controller, the fluid outlet to direct air to the facial region of the user.

In some implementations, determining the position of the head of the user includes determining, in real-time, the position of the head of the user.

In some implementations, determining the position of the head of the user includes determining the position of a nose or mouth of the user.

In some implementations, the method also includes controlling, by the controller, a flow rate of the fan.

In some implementations, controlling the flow rate of the fan comprises controlling the flow rate of the fan at a variable flow rate or a pulsed flow rate.

Implementations of the present disclosure include an air delivery system that includes a fan coupled to a portable article of a user. The air delivery system also includes a fluid conduit fluidly coupled to the fan. The fluid conduit includes a fluid outlet configured to deliver air to a face of the user. The air delivery system also includes a tracking system coupled to the portable article. The tracking system includes at least one motion sensor and a controller configured to control the outlet of the fluid conduit based on information received from the at least one motion sensor. The at least one motion sensor tracks a position of a portion of a head of the user below eyes of the user.

In some implementations, the fluid outlet resides at the portable article away from an ambient breathing proximity of the user. In some implementations, the portable article includes a backpack, in which the fluid outlet is generally flush with a surface of a shoulder strap of the backpack.

In some implementations, the air delivery system also includes an air filter fluidly coupled to the fan. The air filter filters the air circulated through the fluid conduit.

In some implementations, the fluid outlet includes one or more vents and the controller is configured to move the vents to control the fluid outlet to change a direction of the air.

In some implementations, the at least one motion sensor senses motion information representing motion of the jaw of the user. The controller determines a position of the head of the user based on the motion information and the controller controls the outlet based on the determined position of the head.

In some implementations, the tracking system further includes one or more sensors configured to sense ambient condition information representing at least one of 1) wind speed information, 2) wind direction information, or 3) airborne particulate information, and the controller is configured to control, based on the ambient condition information, at least one of the fluid outlet and the fan.

Implementations of the present disclosure also include an air delivery apparatus that includes a fan and a fluid conduit fluidly coupled to the fan. The fluid conduit includes a fluid outlet that resides away from an ambient breathing proximity of a user. The fluid outlet is configured to deliver air to the face of the user. The air delivery apparatus also includes at least one sensor configured to sense motion information of the user. The motion information represents a motion of a lower portion of a face of the user. The air delivery apparatus also includes a controller communicably coupled to the sensor and configured to perform operations including 1) receiving the motion information from the at least one sensor; and 2) controlling, based on the motion information, an orientation of the fluid outlet.

In some implementations, the fluid outlet resides at a surface of a portable article carried by the user, the surface disposed away from the ambient breathing proximity of the user.

In some implementations, the at least one sensor includes multiple sensors configured to sense ambient condition information representing at least one of 1) wind speed information, 2) wind direction information, or 3) airborne particulate information. The controller is configured to control, based on the ambient condition information, at least one of the fluid outlet and the fan.

In some implementations, the at least one sensor includes multiple sensors configured to sense facial information of the user representing the lower portion of the face of the user. The controller is configured to perform operations including 1) determining, based on the facial information of the user and on a mathematical model of the Coand\check{a} effect, a location of a facial region of the user away from a nose and mouth of the user; and 2) controlling the fluid outlet to direct the air to the facial region of the user.

Implementations of the present disclosure also include an air delivery apparatus that includes an oxygen source and a fluid conduit fluidly coupled to the oxygen source. The fluid conduit includes a fluid outlet residing away from an ambient breathing proximity of a user and is configured to deliver air to the face of the user. The air delivery apparatus also includes at least one sensor configured to sense motion information of the user. The motion information represents a motion of a lower portion of a face of the user. The air delivery apparatus also includes a controller communicably coupled to the sensor and configured to perform operations including 1) receiving the motion information from the at least one sensor; and 2) controlling, based on the motion information, an orientation of the fluid outlet.

The present disclosure features an air delivery system that delivers air to a user accurately and efficiently. For example, by tracking the motion of a portion of the user below the eyes of the user, the system can accurately detect the orientation of the head and direct an airflow in a desired direction. The air delivery system can also save energy by controlling a flow rate (e.g., at a variable flow rate, constant flow rate, pulsed flow rate, or combination thereof) of the fan based on environmental conditions and user-related parameters. Additionally, the air delivery system disclosed herein is hidden from view and is implemented in commonly used articles to provide the user a “seamless” experience. Furthermore, the air delivery system resides away from a breathing proximity of the user so that the user does not have to wear anything on or close to the face to receive the air.

The details of one or more implementations of the subject matter described in this disclosure are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a schematic perspective view of a user carrying an air delivery system in a backpack.

FIG. 2 is a schematic front view of the user carrying the air delivery system of FIG. 1.

FIG. 3 is a schematic front view of a user carrying an air delivery system in a jacket.

FIG. 4A is a schematic perspective view of an air delivery system implemented in a hospital bed.

FIG. 4B is a schematic perspective view of an air delivery system implemented in a wheelchair.

FIG. 5 shows a block diagram of an example air delivery system.

FIG. 6 shows a flowchart of an example method of delivering air to a user.

FIG. 7 shows an example controller or control system for an air delivery system according to the present disclosure.

### DETAILED DESCRIPTION OF THE DISCLOSURE

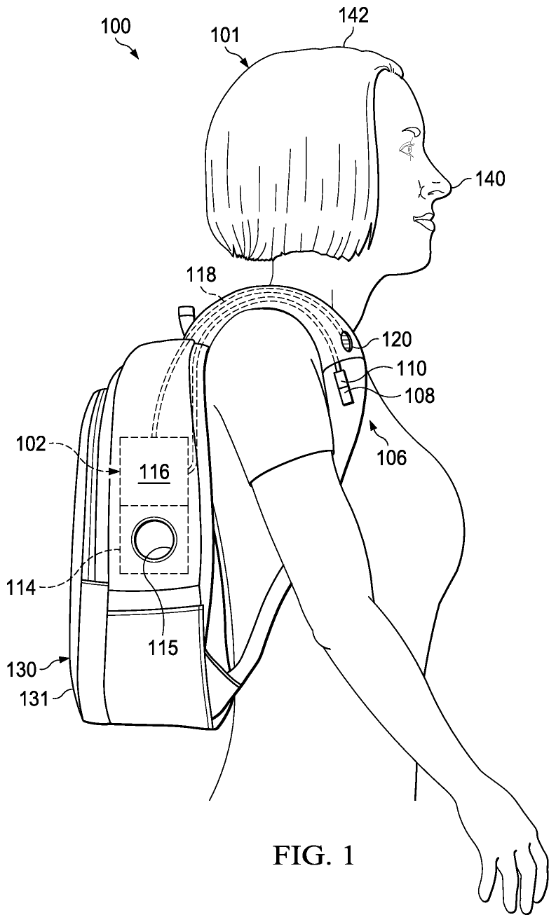

FIG. 1 shows an individual air delivery system 100 that includes an airflow system 102, a fluid conduit 104 fluidly coupled to the airflow system 102, and a tracking system 106. The airflow system 102 is disposed inside a backpack 131 carried by a user 101. The air delivery system 100 is disposed inside the backpack 131, preferably hidden from view. Although FIG. 1 shows the air delivery system 100 implemented in a portable or wearable article 130 such as a backpack, the system 100 can be implemented in non-portable articles or environments such as in hospital beds, wheelchairs, and desks.

The example airflow system 102 includes a fan 114 and an air filter 116. The airflow system 102 is fluidly coupled to a fluid conduit 118 that receives and flows air from the fan 114 to the user 101. The fluid conduit 118 has a fluid outlet 120 that delivers air to the face 140 or near the face 140 of the user 101. All or most of the components of the air delivery system 100 may be disposed inside the backpack 131 and confined or hidden from view such that the backpack 131 shows no apparent modifications. Specifically, the air delivery system 100 may be preferably confined in articles regularly used by the user 101, instead of having a dedicated housing or assembly that is visible and apparent to those around the user 101. In other words, the air delivery system 100 is not invasive or intrusive, but instead, the system 100 is hidden from the user 101 and from others around the user 101. For example, only the fluid outlet 120 of the fluid conduit 118 can be slightly exposed outside the backpack 131. Other components, such as a user interface (e.g., touchscreen or other control device), as well as a colorimetric sensor, may also be exposed or partially exposed outside the backpack 131.

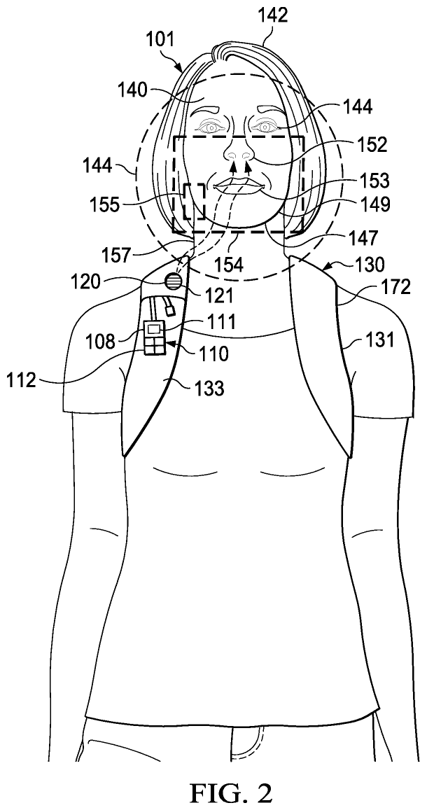

The example implementation of the tracking system 106 includes one or more sensors 110 and a controller 108 that controls the fluid outlet 120 and the fan 114 to change a direction and flow rate of the fan 114. Referring also to FIG. 2, the one or more sensors 110 includes at least one motion sensor or detector 112 that tracks a position of a portion 154 of the head 142 of the user 101, e.g., below the eyes 144 of the user 101. The motion sensor 112 may be, for example, an image detector such as a camera, a radar sensor, or any type of sensor or device that can detect a motion or position of the portion 154 of the head 142 of the user 101, e.g., below the eyes 144 of the user 101. The controller 108 controls the outlet 120 (e.g., the orientation of the fluid outlet) based on information received from the motion sensor 112 to direct the airflow toward the head 142 of the user 101 or toward an ambient breathing proximity 141 of the user 101. The tracking system 106 can include algorithms for facial feature extraction, facial action unit recognition, facial landmark detection, and head pose estimation.

The fluid outlet 120 resides at a location away from the ambient breathing proximity 141 of the user 101. In some implementations, the ambient breathing proximity 141 of the user 101 can be any point in space that is at a distance of less than about 6 inches away from the nose 152 or mouth 153 of the user 101 (e.g., with the head 142 straight). For example, the fluid outlet 120 can reside between 6 to 24 inches away from the nose 152 or mouth 153 of the user.

The fluid outlet 120 can reside at a surface 133 of the portable article 130. In some aspects, the fluid outlet 120 can be generally flush with the surface 133 of a shoulder strap 172 of the backpack 131. In some implementations, the fluid outlet 120 can extend less than about 0.5 inches from the surface 133 of the backpack 131. The fluid outlet 120 can include vents 121 that are controlled by the controller 108 to change a direction of the airflow 135 flowing from the outlet 120. The vents 121 can include horizontal and vertical vents that direct the airflow in multiple directions. In some implementations, the vents 121 (or the fluid outlet) can include a spherical-type vent such as a spherical spout air vent. A small actuator coupled to the vents 121 and controlled by the controller 108 can change the orientation of the vents 121.

As shown in the example implementation of FIG. 1, the air filter 116 is fluidly coupled to the fan 114 and can be located at an air intake or outtake of the fan 114. The fluid conduit 118 flows the filtered air to the face of the user 101. The fan 114 draws ambient air from outside the backpack 131 through an air intake 115, and directs the air toward the fluid conduit 118. The air filter 116, in some aspects, may be a high-efficiency particular air (HEPA) filter, an ultraviolet (UV) filter, an electrostatic filter, an ionizer filter, a cyclonic filter, or a combination thereof. The air filter 116 can include an antimicrobial coating to kill airborne and encrusted microbes, and a photocatalytic coating to neutralize harmful gases. The system 100 can include a sensor (e.g., a colorimetric sensor) to alert the user 101 when the filter cartridge needs replacement and when charged plates (of an electrostatic air filter) need to be cleaned. The colorimetric sensor can also display changes in ambient air quality.

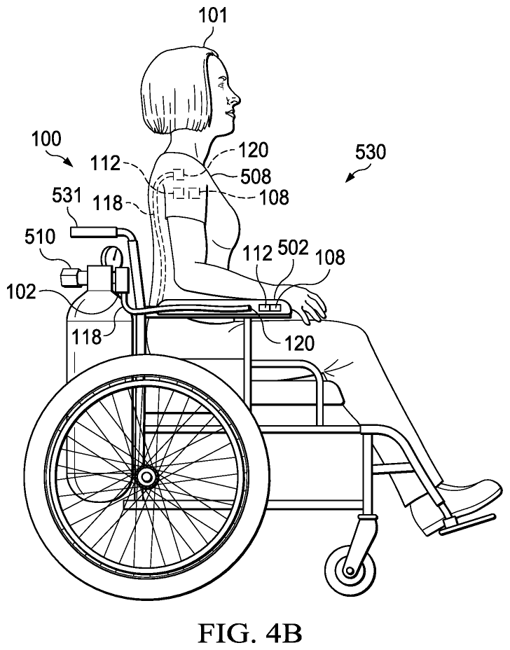

As further described in detail below with respect to FIGS. 2 and 5, the controller 108 (e.g., a processor of the controller 108) can determine, based on the information received from the sensors 110, a position of the head 142 of the user 101, a time of inhalation of the user 101, and ambient conditions surrounding the user 101. The controller 108 uses such determinations and information to control the fluid outlet 120 and the fan 114.

The motion sensor (or detector) 112 senses the motion of the head 142 of the user 101 and sends the motion information to the controller 108. The controller 108 includes or is coupled to a processor 111 that processes the information received from the sensors 110 and determines, based on the sensor information, a direction in which the fluid outlet 120 will deliver the airflow 135. For example, the processor 111 determines a position of the head 142 of the user 101 and, based on the position of the head 142, the processor 111 determines in what direction the fluid outlet 120 will point. The processor 111 sends the information to the controller 108 and the controller 108 controls, based on the information received from the processor 111, the fluid outlet 120 to change a direction of the air. The processor 111 can also cumulatively develop an individual profile that charts the user's exposure to air pollution over time.

The sensors 110 can send the motion information to the controller 108 in real time, and the controller 108 can determine, in real time, the position of the head 142 of the user 101. In example implementations, “real time” means that a duration between receiving an input and processing the input to provide an output can be minimal, for example, in the order of seconds, milliseconds, microseconds, or nanoseconds, sufficiently fast to follow, with the airflow, the position of the head 142 of the user.

The motion information represents the motion of the lower portion 154 of the head 142 of the user 101 below the eyes 144 of the user 101. For example, the motion detector 112 can sense the orientation of the jaw 147, the orientation of the nose 152, or the orientation of the mouth 153 or neck or shoulders of the user 101. In some implementations, the motion detector 112 can track the orientation of the jawline 149 of the user 101. By tracking the motion of the jawline 149, the controller 108 determines the position of the head 142 based on the tracking information received from the detector 112, which includes the position and orientation of the jaw 147 or jawline 149. The jawline 149 can include the curve of the jaw 147 from ear to ear or a portion of the curve of the jaw 147. An advantage of using the jawline 149 as an identifying geometry is the optical visibility of the jawline 149 from below because of the rigid and constant shape of the jaw. This can provide dependable three-dimensional positioning of the head relative to a location of the motion sensor 112. Thus, the controller 108 (with the processor 111) determines, based on the motion information, a position of the head 142 of the user 101, and then controls the fluid outlet 120 (or the airflow system 102 or both) based on the position of the head 142.

The one or more sensors 110 can sense one or more environmental or ambient parameters including, without limitation, humidity, temperature, wind speed, wind direction, air quality, and air particles (e.g., particulate type and density). Thus, the controller 108 receives, from the sensors 110, ambient condition information that represents at least one of wind speed information, wind direction information, and airborne particulate information. The controller 108 then controls based on the ambient condition information, the fluid outlet 120 or the airflow system 102 (e.g., the fan 114) or both. In some implementations, the tracking system 106 can include a global positioning system (GPS) and the controller 108 can be wirelessly connected to the internet or to a cellular network to receive weather information and other environmental or ambient information.

The one or more sensors 110 can also sense breathing information of the user 101 such as the inhalation and exhalation of the user 101. The controller 108 receives the breathing information from the sensors 110 and determines a time of inhalation of the user 101. The controller then controls, based on the time of inhalation, the fluid outlet 120 or the airflow system 102 (e.g., the fan 114) or both. Thus, the controller 108 can control the fluid outlet 120 and the fan 114 based on one or more of 1) the position of the head 142 of the user 101, 2) ambient condition information, and 3) breathing information of the user 101. For example, the controller 108 can control the flow rate of the fan 114 and the orientation of the vents 121 to save power and effectively deliver air to the user 101.

As shown in FIG. 2, the one or more sensors 110 can also sense facial information of the user 101. For example, the one or more sensors 110 can sense biometric information of the user 101 that includes facial features (e.g., three-dimensional facial features) of the user 101 below the eyes 144 of the user 101. The controller 108 can determine, based on the facial information of the user 101 and on a mathematical model of the Coanda effect, a location of a point or facial region 155 of the face 140 of the user 101 spaced from the nose 152 and the mouth 153 of the user 101. The Coanda effect involves air clinging to surfaces and following the contours of an object. In some aspects, the mathematical model of the Coanda effect may be a machine learned model based on a physics model. For example, the model may be a learning simulation that provides, e.g., a single model implementation across a number of physical domains that may involve or include fluids (e.g., airflows), rigid solids, and/or deformable materials that interact with one another.

The facial region 155 is spaced from the nose 152 or mouth 153 of the user 101 a distance enough to allow the air, under the Coanda effect, to ‘attach’ to and flow along the surface of the face 140 of the user 101 to the nose 152 or mouth 153 of the user 101. Upon determining the position of such point 155, the controller 108 controls the fluid outlet 120 (and the fan 114, if necessary) to direct air to the region (or point) 155 of the face 140 of the user 101. Thus, using the Coanda effect to deliver air can help deliver air reliably to positions that might not have line-of-sight from the fluid outlet 120. In some implementations, the face 140 can include the neck 157 of the user 101 and the facial region 155 can be a region on the neck 157 of the user 101. For example, the region 155 can be spaced between about 3 to 8 inches from the nostrils or mouth of the user.

The air delivery system 100 can also include other features to improve the user experience. For example, the air delivery system 100 can include a noise-cancelling device (e.g., a noise cancelling speaker) disposed at the airflow system 102 or the tracking system 106 to reduces fan blade noise and other noises of the air delivery system. The air delivery system 100 can also include a fluid emitter (e.g., a chemical emitter) and an electronic nose to detect odors near the user 101. The electronic nose and the fluid emitter can be part of the tracking system 106. The fluid emitter can deliver to the user 101, without limitation, a cooling mist, insect repellent, and aerosolized drugs (e.g., hypertonic saline to clean the respiratory tract). The fluid emitter can be communicatively coupled to the electronic nose to deliver, based on odor information received from the electronic nose, a desired amount of scents (e.g., olfactory receptor antagonists to turn off smell perception of the user 101 when the user 101 is exposed to unpleasant odors).

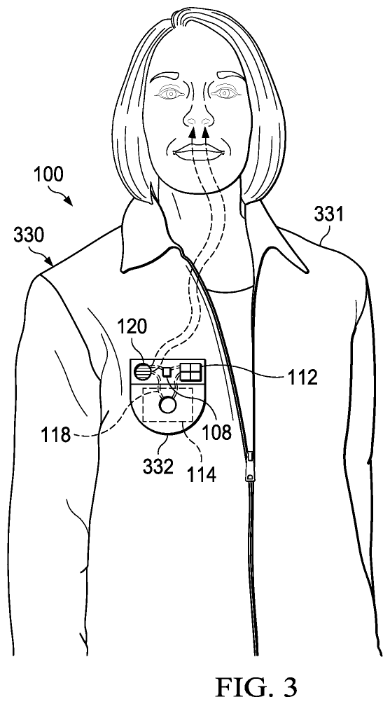

FIG. 3 shows the air delivery system 100 implemented in a clothing article 330 such as a jacket 331. Most or all of the air delivery system 100 can be disposed inside a pocket 332 of the jacket 331. Specifically, the controller 108, the sensors 112, and the fluid conduit 118 are disposed inside the pocket 332, with the fluid outlet 120 exposed through the pocket 332. The fan 114 can reside in the pocket 332 or at a different location of the jacket 331 (e.g., a different pocket). In some implementations, the air delivery system 100 can reside at a different location such as around the shoulder area or the arm.

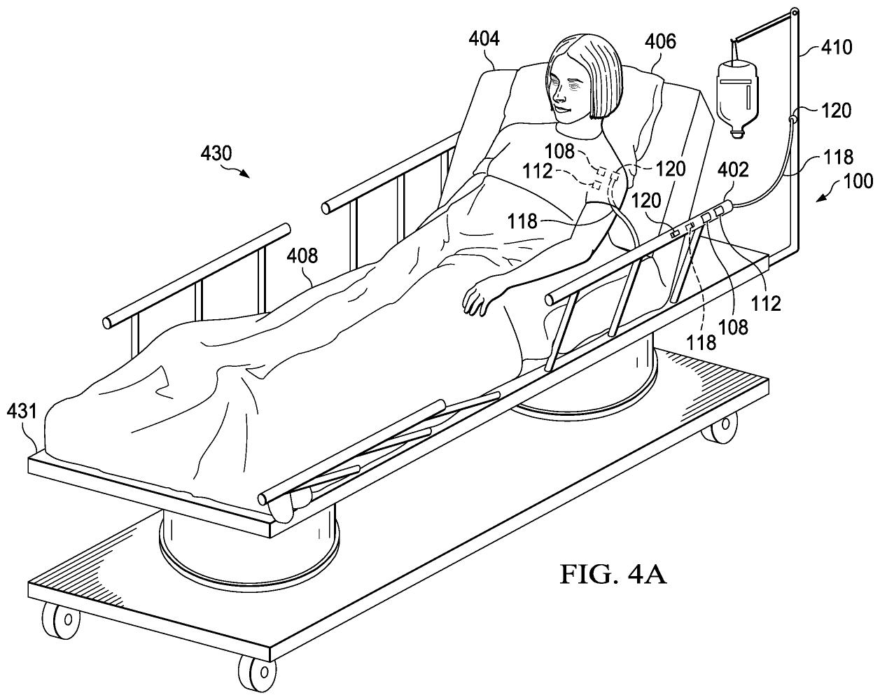

FIGS. 4A and 4B show the air delivery system 100 implemented in non-portable objects 430 and 530. FIG. 4A illustrates the air delivery system implemented in a hospital bed 431. For example, the air delivery system 100 (or a portion of the system 100) can be attached to a side rail 402 of the bed, to the mattress 404, or to a pillow 406. For example, the controller 108, the sensors 112, and the fluid conduit 118 can be disposed inside the side rail 402; with the fluid outlet 120 and the sensors 112 exposed through an aperture of the side rail 402. The fluid conduit 118 may also be attached to an exterior surface of the side rail 402. In some implementations, all or part of the air delivery system 100 can be attached to a pole 410 (e.g. an intravenous fluid stand) near the hospital bed 431. In some implementations, all or part of the air delivery system 100 can be attached to a hospital gown or a blanket 408 covering the patient.

FIG. 4B shows the air delivery system 100 implemented in a wheelchair 531. The air delivery system 100 (or a portion of the system 100) can be attached to an armrest 502 of the wheelchair 531. For example, the controller 108, the sensors 112, the fluid conduit 118, and the fluid outlet 120 can be disposed inside or outside the side rail 402. In some implementations, the air delivery system 100 (or part of the system 100) can be attached to a hospital gown or a clothing article 508 worn by the user 101. In some examples, the airflow system 102 can receive oxygen from an oxygen tank 510 (e.g., with pressurized oxygen) mounted to the back of the wheelchair 531. The airflow system 102 can flow the oxygen (e.g., without a fan or filter, using a valve to regulate oxygen flow) to the user 101 or blend the oxygen with the airflow of the system 102 to deliver an airflow to the user 101. Such implementation can eliminate the need of using nasal cannulas, masks, or other facial devices used to deliver oxygen to a user. Although FIG. 4B illustrates an implementation of the air delivery system 100 in a wheelchair 531, other mobile vehicles, such as scooter, baby carriages, or strollers may also be implemented with the air delivery system similarly as the wheelchair 531.

FIG. 5 shows a block diagram of an example implementation of the air delivery system 100. The air delivery system 100 includes the airflow system 102 fluidly coupled to the fluid outlet 120 and the tracking system 106 operationally coupled to the fluid outlet 120. The tracking system 106 includes the controller 108, the processor 111, and the sensors 110. The controller 108 is communicatively coupled to the sensors 110 and operationally coupled to the fluid outlet 120 to control the fluid outlet 120.

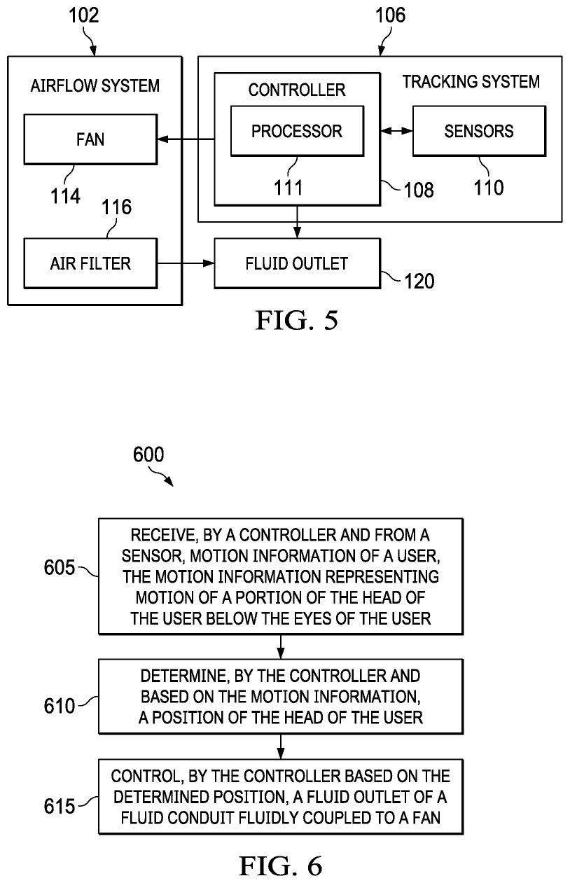

FIG. 6 shows a flow chart of a method 600 of delivering air to a user. The method includes receiving, by a controller and from a sensor, motion information of a user (605). The motion information represents motion of a portion of the head of the user below the eyes of the user. The method also includes determining a position of the head based on the information (610). The method also includes controlling, by the controller based on the determined position, a fluid outlet of a fluid conduit fluidly coupled to a fan (615).

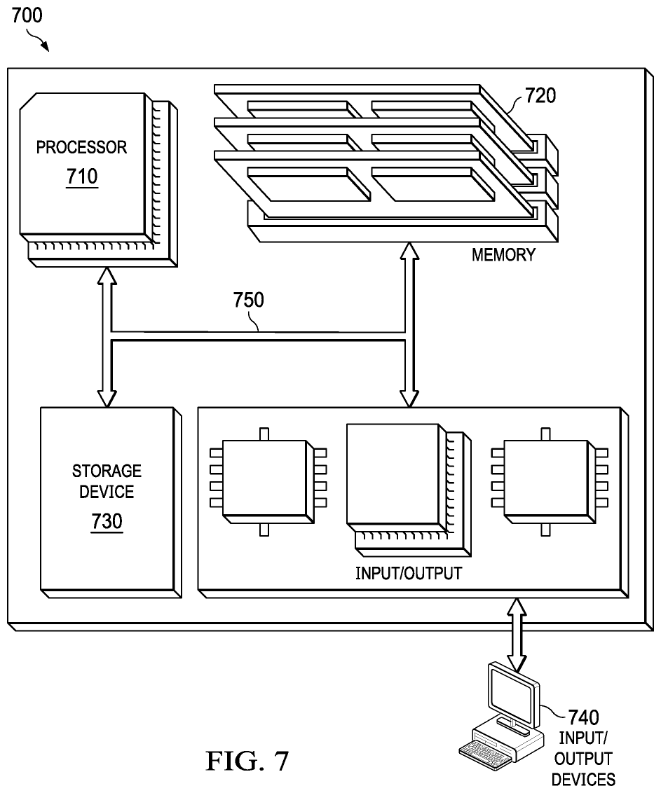

FIG. 7 is a schematic illustration of an example control system or controller for an air delivery system according to the present disclosure. For example, the controller 700 may include or be part of the controller 108 shown in FIG. 5. The controller 700 is intended to include various forms of digital computers, such as printed circuit boards (PCB), processors, digital circuitry, or otherwise. Additionally the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The controller 700 includes a processor 710, a memory 720, a storage device 730, and an input/output device 740. Each of the components 710, 720, 730, and 740 are interconnected using a system bus 750. The processor 710 is capable of processing instructions for execution within the controller 700. The processor may be designed using any of a number of architectures. For example, the processor 710 may be a CISC (Complex Instruction Set Computers) processor, a RISC (Reduced Instruction Set Computer) processor, or a MISC (Minimal Instruction Set Computer) processor.

In one implementation, the processor 710 is a single-threaded processor. In another implementation, the processor 710 is a multi-threaded processor. The processor 710 is capable of processing instructions stored in the memory 720 or on the storage device 730 to display graphical information for a user interface on the input/output device 740.

The memory 720 stores information within the controller 700. In one implementation, the memory 720 is a computer-readable medium. In one implementation, the memory 720 is a volatile memory unit. In another implementation, the memory 720 is a non-volatile memory unit.

The storage device 730 is capable of providing mass storage for the controller 700. In one implementation, the storage device 730 is a computer-readable medium. In various different implementations, the storage device 730 may be a floppy disk device, a hard disk device, an optical disk device, or a tape device.

The input/output device 740 provides input/output operations for the controller 700. In one implementation, the input/output device 740 includes a keyboard and/or pointing device. In another implementation, the input/output device 740 includes a display unit for displaying graphical user interfaces.

A number of embodiments have been described. Nevertheless, it will be understood that various modifications may be made without departing from the spirit and scope of what is described. For example, the steps of exemplary operations described herein may be performed in other orders than those described, some steps may be removed, and other steps may be added. Accordingly, other embodiments are within the scope of the following claims.

What is claimed is:

1. A method comprising:
receiving, by a controller and from a sensor, motion information of a user, the motion information representing motion of a portion of a head of the user below eyes of the user, the sensor and the controller coupled to a portable article of the user;
determining, by the controller and based on the motion information, a position of the head of the user; and
controlling, by the controller based on the determined position, a fluid outlet of a fluid conduit that is fluidly coupled to, and configured to receive air from, a fan that is coupled to the portable article of the user.

2. The method of claim 1, wherein receiving the motion information comprises receiving the motion information from a motion detector configured to sense an orientation of a jaw of the user.

3. The method of claim 1, wherein controlling the fluid outlet comprises controlling vents of the fluid outlet to change a direction of air flowing from the fluid outlet.

4. The method of claim 1, further comprising:
receiving, from one or more sensors, ambient condition information representing at least one of 1) wind speed information, 2) wind direction information, or 3) airborne particulate information; and
controlling, by the controller and based on the ambient condition information, at least one of the fluid outlet and the fan.

5. The method of claim 4, further comprising:
receiving, from the one or more sensors, breathing information of the user;
determining, by the controller and based on the breathing information, a time of inhalation of the user; and
controlling, by the controller, based on the ambient condition information and on the time of inhalation, at least one of the fluid outlet and the fan.

6. The method of claim 1, further comprising:
receiving, from one or more sensors, facial information of the user representing facial features of the user below the eyes of the user;
determining, by the controller, based on the facial information of the user and on a mathematical model of the Coand\check{a} effect, a location of a facial region of the user away from a nose and a mouth of the user; and
controlling, by the controller, the fluid outlet to direct air to the facial region of the user.

7. The method of claim 1, wherein determining the position of the head of the user comprises determining, in real-time, the position of the head of the user.

8. The method of claim 1, wherein determining the position of the head of the user comprises determining the position of a nose or mouth of the user.

9. The method of claim 1, further comprising controlling, by the controller, a flow rate of the fan.

10. The method of claim 9, wherein controlling the flow rate of the fan comprises controlling the flow rate of the fan at a variable flow rate or a pulsed flow rate.

11. An air delivery system comprising:
a fan configured to be coupled to a portable article of a user;
a fluid conduit fluidly coupled to the fan, the fluid conduit comprising a fluid outlet configured to deliver air to a face of the user; and
a tracking system configured to be coupled to the portable article, the tracking system comprising at least one motion sensor and a controller configured to control the outlet of the fluid conduit based on information received from the at least one motion sensor, the at least one motion sensor configured to track a position of a portion of a head of the user below eyes of the user.

12. The air delivery system of claim 11, wherein the fluid outlet resides at the portable article away from an ambient breathing proximity of the user.

13. The air delivery system of claim 12, wherein the portable article comprises a backpack, and wherein the fluid outlet is generally flush with a surface of a shoulder strap of the backpack.

14. The air delivery system of claim 11, further comprising an air filter fluidly coupled to the fan, the air filter configured to filter the air circulated through the fluid conduit.

15. The air delivery system of claim 11, wherein the fluid outlet comprises one or more vents, and wherein the controller is configured to move the vents to control the fluid outlet to change a direction of the air.

16. The air delivery system of claim 11, wherein the at least one motion sensor is configured to sense motion information representing motion of a jaw of the user, the controller configured to determine a position of the head of the user based on the motion information and the controller configured to control the outlet based on the determined position of the head.

17. The air delivery system of claim 11, wherein the tracking system further comprises one or more sensors configured to sense ambient condition information representing at least one of 1) wind speed information, 2) wind direction information, or 3) airborne particulate information, and the controller is configured to control, based on the ambient condition information, at least one of the fluid outlet and the fan.

18. An air delivery apparatus comprising:
an air source;
a fluid conduit fluidly coupled to the air source, the fluid conduit comprising a fluid outlet residing away from an ambient breathing proximity of a user and configured to deliver air to a face of the user;
at least one sensor configured to sense motion information of the user, the motion information representing a motion of a lower portion of a face of the user; and
a controller communicably coupled to the sensor and configured to perform operations comprising:
receiving the motion information from the at least one sensor, and
controlling, based on the motion information, an orientation of the fluid outlet.

19. The air delivery apparatus of claim 18, wherein the air source comprises a pressurized oxygen tank.

20. The air delivery apparatus of claim 18, wherein the fluid outlet resides at a surface of a portable article carried by the user, the surface disposed away from the ambient breathing proximity of the user.

21. The air delivery apparatus of claim 18, wherein the air source comprises a fan fluidly coupled to an ambient environment.

22. The air delivery apparatus of claim 21, wherein the at least one sensor comprises a plurality of sensors configured to sense ambient condition information representing at least one of 1) wind speed information, 2) wind direction information, or 3) airborne particulate information, and wherein the controller is configured to control, based on the ambient condition information, at least one of the fluid outlet or the fan.

23. The air delivery apparatus of claim 18, wherein the at least one sensor comprises a plurality of sensors configured to sense facial information of the user representing the lower portion of the face of the user, and the controller is configured to perform operations comprising:
determining, based on the facial information of the user and on a mathematical model of the Coand\check{a} effect, a location of a facial region of the user away from a nose and mouth of the user; and
controlling the fluid outlet to direct the air to the facial region of the user.
